(19) United States
(12) Patent Application Publication
(10) Pub. No.: US 2015/0376597 A1
(43) Pub. Date: Dec. 31, 2015

(54) Novel methods for the targeted introduction of viruses into cells

(71) Applicants: FRIEDRICH MIESCHER INSTITUTE FOR BIOMEDICAL RESEARCH (FMI), Basel (CH); EIDG. TECHNISCHE HOCHSCHULE (ETH), Zurich (CH)

(72) Inventors: Kamill BALINT, Basel (CH); Daniel Jobst MÜLLER, Basel (CH); Botond ROSKA, Basel (CH); Rajib SCHUBERT, Basel (CH)

(73) Assignees: FRIEDRICH MIESCHER INSTITUTE FOR BIOMEDICAL RESEARCH (FMI), Basel (CH); Eldg. Technische Hochschule (ETH), Zürich (CH)

(21) Appl. No.: 14/766,165

(22) PCT Filed: Feb. 6, 2014
(86) PCT No.: PCT/IB2014/058834

§ 371 Date: Aug. 6, 2015

(30) Foreign Application Priority Data
Feb. 8, 2013 (EP) 13154724.2

(51) Int. Cl. C12N 11/14; C12N 7/00

(52) U.S. Cl. CPC C12N 11/14; C12N 7/00; C12N 2799/00

(57) ABSTRACT

The present invention provides a method for the targeted infection of cells. Said method is characterized in that it comprises the step of contacting the cell with a virus attached to a support. The present invention also encompasses a support to which a molecule binding specifically to a molecule present on the surface of a virus is attached, said molecule binding specifically to a molecule present on the surface of said virus being optionally attached to the support through a linking moiety.

### FIELD OF THE INVENTION

The present invention provides novel methods and tools for the introduction of viruses into cells.

### BACKGROUND OF THE INVENTION

Most experimental procedures relying on the infection of a cell by a virus are done using bulk infections. Whereas, it is possible to restrict the expression of the viral protein so specific cell populations using specific promoters, there is still a need in the art for a targeted introduction of viruses into discrete, predefined cells.

For example, investigations on single neurons and on their connected brain circuit led to major insights to brain function. Single neuron recordings opened the way to understand brain computations. Viral labeling of neurons and their connected circuit on the other hand revolutionized neuroscience by providing ways to bring genetic tools such as green fluorescent protein, calcium sensors or optogenetic probes to nerve cells themselves or the nerve cells and their connected elements. However no current technology exists to pick up a virus and deliver it into a single nerve cell which limits our ability to deliver genes to identified neurons and their connected circuits.

Atomic force microscopes (AFM) have been extensively applied to image and to mechanically manipulate various viruses (see, e.g., Atomic force microscopy in imaging of viruses and virus-infected cells. Kuznetsov Y G, McPherson A. Microbiol Mol Biol Rev. 2011 June; 75(2):268-85). AFM has also been applied to characterize the interaction between antigen and genes and between receptor and ligands. Recently, an AFM approach has been invented to pick up a single molecule by the tip of the AFM cantilever and to transport and to deliver this molecule at a given area (Single-molecule cut-and-paste surface assembly. Kufer S K, Puchner E M, Gumpp H, Liedl T, Gaub H E. Science. 2008 Feb. 1; 319(5863):594-6.). In another recent application, an AFM, using a fluid channel, has been designed to transport viruses onto cells (Cooperative vaccinia infection demonstrated at the single-cell level using FluidFM. Stiefel P, Schmidt F I, Dörig P, Behr P, Zambelli T, Vorholt J A, Mercer J. Nano Lett. 2012 Aug. 8; 12(8):4219-27.).

There is however a need in the art for simple tools which will allow a specific, targeted, viral infection of single specific cells.

### SUMMARY OF THE INVENTION

As explained herein-above, there was a desire in the art for simple tools which allow the targeted/specific viral infection of single specific cells.

The investors have now found out that a relatively simple and robust setting allows solving this need.

The present invention hence provides a method of infecting a cell with a virus characterized in that it comprises the step of contacting the cell with a virus attached to a support.

In some embodiments, the virus is attached to the support through a molecule binding specifically to a molecule present on the surface of said virus. For instance this molecule binding specifically to a molecule present on the surface of said virus can be a monoclonal antibody, a polyclonal antibody, an antibody fragment having a specific binding activity, e.g. F(ab′)2, Fab′, Fab or Fv, a chimeric antibody, e.g. humanized antibody, a scFv, an aptamers or CDRs grafted onto alternative scaffold.

Said molecule binding specifically to a molecule present on the surface of said virus can be attached directly to the support, or can be attached to the support through a linking moiety, for instance a polyethyleneglycol (PEG), polypeptide, a sugar, a nucleic acids, a rod or an extended fiber, e.g. a carbon nanotubes, or combinations thereof.

The viruses used for the present invention can be any viruses, for example, adeno-associated viruses (AAV), pseudorabies viruses (PRV), lentiviruses, herpes viruses or rabies viruses.

In some embodiments, the molecule present on the surface of the virus which is recognized by the can be a normal viral coat protein or a viral lipid molecule. In some embodiments, said viral coat protein or a viral lipid molecule can be modified in order to e.g. influence the specificity of the molecule binding specifically to a molecule present on the surface of said virus and/or the binding force of this interaction. In some embodiments, exogenous molecules, which are recognized and bound by the specifically-binding molecule can be expressed on the surface of the virus.

In some embodiments, the method of the invention will comprise the step of physically touching the cell to be infected with the support to which the virus is attached. In other embodiments, the support will not touch the cell but merely bring the virus within a distance allowing an interaction between the virus and the cell.

The support itself can be for example bound to a micromanipulator in order to increase the precision of the method.

In some embodiments, the support is the tip of a microelectrode or of a pipette, a bead or microscopic particle, or is a microscopic or nanoscopic device such as a cantilever.

The method can be also be carried out by a robot in a semi or fully automated fashion.

In some embodiments, at least two different viruses are attached to the support contacting the cell. This can easily be achieved by using bi-valent virus binding molecules or by attaching different virus-binding molecules to the support. The viruses used can be viruses complementing each other, e.g. inactivated viruses and rescue viruses. In other embodiments, said different viruses will be used sequentially.

The present invention also provides a support as defined herein-above, i.e. a support to which a molecule binding specifically to a molecule present on the surface of a virus is attached, said molecule binding specifically to a molecule present on the surface of said virus being optionally attached to the support through a linking moiety.

For instance this molecule binding specifically to a molecule present on the surface of said virus can be a monoclonal antibody, a polyclonal antibody, an antibody fragment having a specific binding activity, e.g. F(ab′)2, Fab′, Fab or Fv, a chimeric antibody, e.g. humanized antibody, a scFv, an aptamers or CDRs grafted onto alternative scaffold.

Said molecule attached to the support and binding specifically to a molecule present on the surface of said virus can be attached directly to the support, or can be attached to the support through a linking moiety, for instance a polyethyleneglycol (PEG), polypeptide, a sugar, a nucleic acids, a rod or an extended fiber, e.g. a carbon nanotubes, or combinations thereof.

The viruses recognized and bound by said molecule attached to the support and binding specifically to a molecule present on the surface of the virus can be any viruses, for example, adeno-associated viruses (AAV), pseudorabies viruses (PRV), lentiviruses, herpes viruses or rabies viruses. In some embodiments, the molecule present on the surface of the virus which is recognized by the can be a normal viral coat protein or a viral lipid molecule. In some embodiments, said viral coat protein or a viral lipid molecule can be modified in order to e.g. influence the specificity of the molecule binding specifically to a molecule present on the surface of said virus and/or the binding force of this interaction. In some embodiments, exogenous molecules, which are recognized and bound by the specifically-binding molecule can be expressed on the surface of the virus.

The support itself can be for example bound to a micromanipulator in order to increase the precision of the method. In some embodiments, the support is the tip of a microelectrode or of a pipette, a bead or microscopic particle, or is a microscopic or nanoscopic device such as a cantilever.

In some embodiments, the support is part of a semi or fully automated fashion.

In some embodiments, at least two different viruses are attached to the support contacting the cell.

Moreover, the scope of the present invention also includes a kit for the targeted/specific viral infection of single specific cells, said kit comprising a support of the invention.

### DESCRIPTION OF THE FIGURES

Outline of mechanically addressing a virus to a single cell and subsequent viral infection of the cell. (A) Schematic diagram of SADΔG-RFP virus particles immobilized on an AFM cantilever (FIG. 1) and patch pipette (FIG. 2). In a first step (1) the virus is attached to cantilever or glass pipette using a heterobifunctional cross-linker. In the second step (2) the cantilever or glass pipette is used to bring the virus into contact with a cell. In the third step (3) the virus is held in contact with the cell surface so that the virus will bind to the cell surface. In the fourth step (4), the cantilever or glass pipette is withdrawn. If the interactions (bonds) formed between virus and cell surface are stronger than the bond (interaction) of the virus to the cantilever or glass pipette, the virus remains bound at the cell surface. Once attached to the cell surface and released from the cantilever or pipette the virus will be internalized by the cell and will infect the cell. For improved control of the transfection process the individual steps may be monitored using a combination of fluorescence, differential interference and phase contrast microscopy.

FIG. 3 shows the expression of red fluorescence protein (RFP) after virus infection, which indicates subsequent selective infection of a single BHK cell (highlighted at dashed square). The left image of FIG. 3 shows the cultured BHK cells imaged by optical microscopy and the right image shows the fluorescence image of the RFP transfected cell.

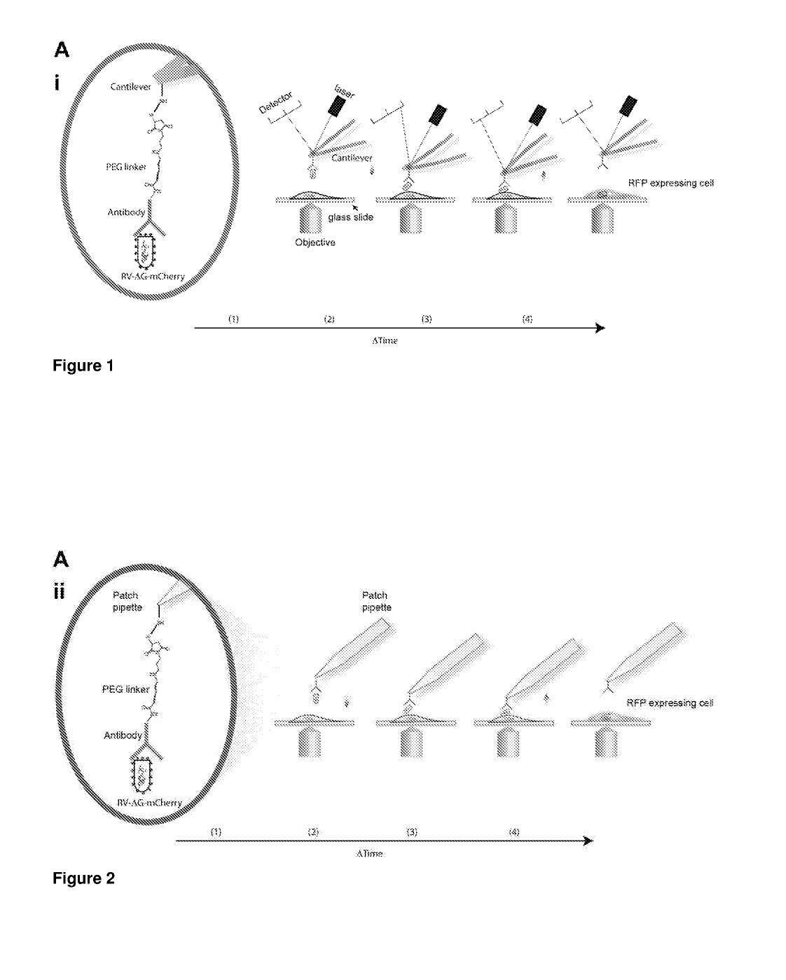

FIG. 4 shows the expression of RFP after virus infection, which indicates subsequent selective infection of a cortical neuron (highlighted at dashed square). The left image of FIG. 4 shows neurons imaged by optical microscopy and the right image shows the fluorescence image of the RFP transfected neuron.

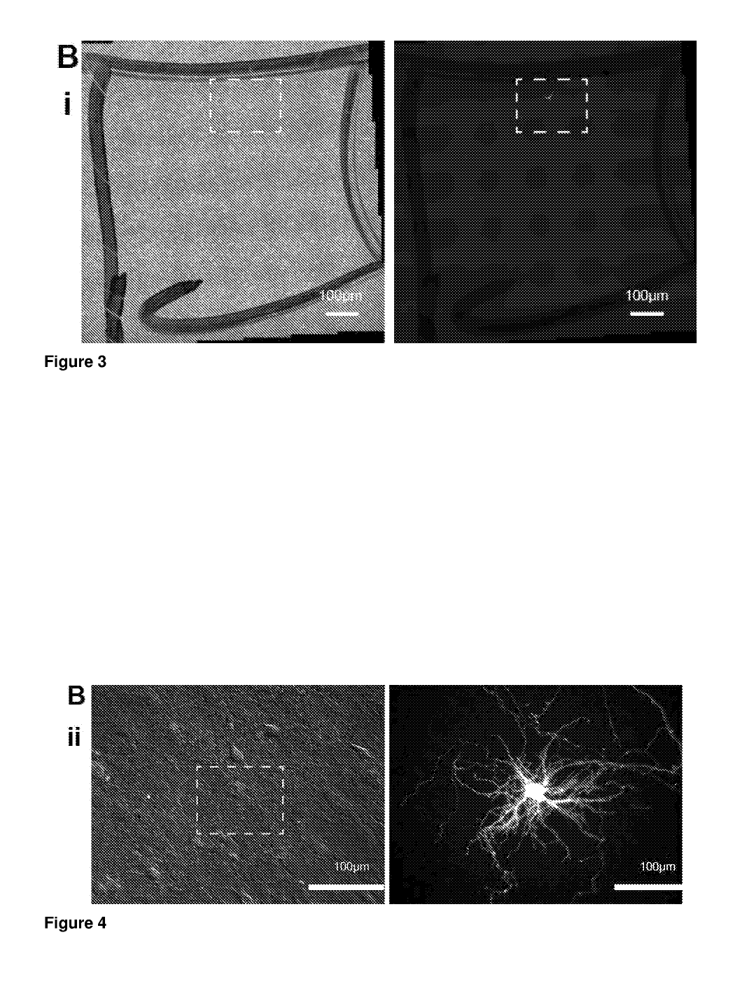

### DETAILED DESCRIPTION OF THE INVENTION

As explained herein-above, there was a desire in the art for simple tools which allow the targeted/specific viral infection of single specific cells.

The investors have now found out that a relatively simple and robust setting allows solving this need.

The present invention hence provides a method of infecting a cell with a virus characterized in that it comprises the step of contacting the cell with a virus attached to a support.

In some embodiments, the virus is attached to the support through a molecule binding specifically to a molecule present on the surface of said virus. For instance this molecule binding specifically to a molecule present on the surface of said virus can be a monoclonal antibody, a polyclonal antibody, an antibody fragment having a specific binding activity, e.g. F(ab′)2, Fab′, Fab or Fv, a chimeric antibody, e.g. humanized antibody, a scFv, an aptamers or CDRs grafted onto alternative scaffold.

Said molecule binding specifically to a molecule present on the surface of said virus can be attached directly to the support, or can be attached to the support through a linking moiety, for instance a polyethyleneglycol (PEG), polypeptide, a sugar, a nucleic acids, a rod or an extended fiber, e.g. a carbon nanotubes, or combinations thereof.

The viruses used for the present invention can be any viruses, for example, adeno-associated viruses (AAV), pseudorabies viruses (PRV), lentiviruses, herpes viruses or rabies viruses.

In some embodiments, the molecule present on the surface of the virus which is recognized by the can be a normal viral coat protein or a viral lipid molecule. In some embodiments, said viral coat protein or a viral lipid molecule can be modified in order to e.g. influence the specificity of the molecule binding specifically to a molecule present on the surface of said virus and/or the binding force of this interaction. In some embodiments, exogenous molecules, which are recognized and bound by the specifically-binding molecule can be expressed on the surface of the virus.

In some embodiments, the method of the invention will comprise the step of physically touching the cell to be infected with the support to which the virus is attached. In other embodiments, the support will not touch the cell but merely bring the virus within a distance allowing an interaction between the virus and the cell.

The support itself can be for example bound to a micromanipulator in order to increase the precision of the method.

In some embodiments, the support is the tip of a microelectrode or of a pipette, a bead or microscopic particle, or is a microscopic or nanoscopic device such as a cantilever.

The method can be also be carried out by a robot in a semi or fully automated fashion.

In some embodiments, at least two different viruses are attached to the support contacting the cell. This can easily be achieved by using bi-valent virus binding molecules or by attaching different virus-binding molecules to the support. The viruses used can be viruses complementing each other, e.g. inactivated viruses and rescue viruses. In other embodiments, said different viruses will be used sequentially.

The present invention also provides a support as defined herein-above, i.e. a support to which a molecule binding specifically to a molecule present on the surface of a virus is attached, said molecule binding specifically to a molecule present on the surface of said virus being optionally attached to the support through a linking moiety.

For instance this molecule binding specifically to a molecule present on the surface of said virus can be a monoclonal antibody, a polyclonal antibody, an antibody fragment having a specific binding activity, e.g. F(ab′)2, Fab′, Fab or Fv, a chimeric antibody, e.g. humanized antibody, a scFv, an aptamers or CDRs grafted onto alternative scaffold.

Said molecule attached to the support and binding specifically to a molecule present on the surface of said virus can be attached directly to the support, or can be attached to the support through a linking moiety, for instance a polyethyleneglycol (PEG), polypeptide, a sugar, a nucleic acids, a rod or an extended fiber, e.g. a carbon nanotubes, or combinations thereof.

The viruses recognized and bound by said molecule attached to the support and binding specifically to a molecule present on the surface of the virus can be any viruses, for example, adeno-associated viruses (AAV), pseudorabies viruses (PRV), lentiviruses, herpes viruses or rabies viruses. In some embodiments, the molecule present on the surface of the virus which is recognized by the can be a normal viral coat protein or a viral lipid molecule. In some embodiments, said viral coat protein or a viral lipid molecule can be modified in order to e.g. influence the specificity of the molecule binding specifically to a molecule present on the surface of said virus and/or the binding force of this interaction. In some embodiments, exogenous molecules, which are recognized and bound by the specifically-binding molecule can be expressed on the surface of the virus.

The support itself can be for example bound to a micromanipulator in order to increase the precision of the method. In some embodiments, the support is the tip of a microelectrode or of a pipette, a bead or microscopic particle, or is a microscopic or nanoscopic device such as a cantilever.

In some embodiments, the support is part of a semi or fully automated fashion.

In some embodiments, at least two different viruses are attached to the support contacting the cell.

Moreover, the scope of the present invention also includes a kit for the targeted/specific viral infection of single specific cells, said kit comprising a support of the invention.

These and other aspects of the present invention should be apparent to those skilled in the art, from the teachings herein.

For convenience, the meaning of certain terms and phrases employed in the specification, examples, and appended claims are provided below.

The singular forms “a,” “an,” and “the” include plural reference unless the context clearly dictates otherwise.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected. Examples of viruses are adeno-associated viruses, pseudorabies viruses, lentiviruses, herpes viruses, alphaherpesviruses and rabies viruses.

Some viruses can be transsynatptic viruses. The term “transsynaptic virus” as used herein refers to viruses able to migrate from one neurone to another connecting neurone through a synapse. Examples of such transsynaptic virus are rhabodiviruses, e.g. rabies virus, and alphaherpesviruses, e.g. pseudorabies or herpes simplex virus.

The term “virus” and “transsynaptic virus” as used herein also encompasses viral sub-units having by themselves the capacity to infect cells, and, in the case of transsynatptic viruses, migrate from one neurone to another connecting neurone through a synapse, and biological vectors, such as modified viruses, incorporating such a sub-unit and demonstrating a capability of infecting cells and, in the case of transsynatptic viruses, migrating from one neurone to another connecting neurone through a synapse.

Transsynaptic migration can be either anterograde or retrograde. During a retrograde migration, a virus will travel from a postsynaptic neuron to a presynaptic one. Accordingly, during anterograde migration, a virus will travel from a presynaptic neuron to a postsynaptic one.

“Polynucleotide” and “nucleic acid”, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical.” The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarities (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Cell type” refers to a cell from a given source (e.g., tissue or organ) or a cell in a given state of differentiation, or a cell associated with a given pathology or genetic makeup.

“Polypeptide” and “protein”, used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression product” or “gene product” is a biomolecule, such as a protein or mRNA, that is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

The term “functional equivalent” is intended to include the “fragments”, “mutants”, “derivatives”, “alleles”, “hybrids”, “variants”, “analogs”, or “chemical derivatives” of the native gene or virus.

“Isolated” refers to a polynucleotide, a polypeptide, an immunoglobulin, a virus or a host cell that is in an environment different from that in which the polynucleotide, the polypeptide, the immunoglobulin, the virus or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Stringent conditions” refers to conditions under which a probe may hybridize to its target polynucleotide sequence, but to no other sequences. Stringent conditions are sequence-dependent (e. g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e. g., 10 to 50 nucleotides).

Stringent conditions may also be achieved with the addition of destabilizing agents, such as formamide.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are preferably from about 15 nt to about 150 nt, more preferably from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e. g., hexose phosphodiester; peptide nucleic acids). The modifications may be internal (single or repeated) or at the end (s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e. g., 3′-substituted ribonucleotides or deoxyribonucleotide monomers), any of which are bound together via 5′ to 3′ linkages.

“Biomolecular sequence” or “sequence” refers to all or a portion of a polynucleotide or polypeptide sequence.

The term “detectable” refers to a polynucleotide expression pattern which is detectable via the standard techniques of polymerase chain reaction (PCR), reverse transcriptase-(RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression levels it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein, polypeptide, or amino acid may also refer to the specific substructure of a larger protein to which the protein-capture agent is directed or to the overall structure (e. g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence.

“BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence”, as understood herein, is a group of assembled overlapping sequences, particularly between sequences in one or more of the databases of the invention.

The term “a molecule binding specifically to a molecule present on the surface of a virus” encompasses antibodies.

Moreover, for the purpose of the present invention, both terms “antibody” and “a molecule binding specifically to a molecule present on the surface of a virus” will be used interchangeably. This is however not to be construed as a limitation of the term “a molecule binding specifically to a molecule present on the surface of a virus” to antibodies only,

An antibody (or molecule binding specifically to a molecule present on the surface of a virus) as used in the present invention will specifically bind to a molecule present on the surface of said virus for example a viral coat protein. This term also embraces active fragments of antibodies. An active fragment means a fragment of an antibody having activity of antigen-antibody reaction. Specifically named, these are active fragments, such as F(ab′)2, Fab′, Fab, and Fv. For example, F(ab′)2 results if an antibody is digested with pepsin, and Fab results if digested with papain. Fab′ results if F(ab′)2 is reduced with a reagent such as 2-mercaptoethanol and alkylated with monoiodoacetic acid. Fv is a mono active fragment where the variable region of heavy chain and the variable region of light chain are connected with a linker. Chimeric antibodies are also encompassed. A chimeric antibody is obtained by conserving these active fragments and substituting the fragments of another animal for the fragments other than these active fragments. If needed, humanized antibodies are also envisioned. For the purpose of this invention, the term “antibody” also encompasses scFv and antibody-like molecules able to specifically bind specifically to a molecule present on the surface of the virus, e.g. aptamers of CDRs grafted onto alternative scaffold, which are well-known to the skilled person.

The term “epitopes” as used herein, refers to portions of a polypeptide having antigenic or immunogenic activity in an animal, in some embodiments, a mammal, for instance in a human. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, the part of molecule present on the surface of a virus recognized by an antibody. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CHI, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Antibodies as used herein include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. Antibodies are usually immunoglobulin molecules and immunologically active portions of immunoglobulin molecules, i.e., molecules that contain an antigen binding site that immunospecifically binds an antigen. For the purpose of the present invention, the immunoglobulin molecules can be of any type (e.g., IgG, IgE, IgM, IgD, IgA and IgY), class (e.g., IgGI, IgG2, IgG3, IgG4, IgAI and IgA2) or subclass of immunoglobulin molecule.

Also included in the definition of “antibodies” are antigen-binding fragments comprising any combination of variable region(s) with a hinge region, CH1, CH2, and CH3 domains. The antibodies to be used in the present invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies used in the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992). Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide.

The viruses used for in the present invention may carry labels, which label will be expressed by the infected cell. “Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

A “fluorescent label” refers to any label with the ability to emit light of a certain wavelength when activated by light of another wavelength.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. In some of the embodiments of the present invention, fluorescence will be detected in a qualitative manner. In other words, either the fluorescent marker is present, indicating that the recombinant fusion protein is expressed, or not. For other instances, the fluorescence can be determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.). An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

The “green fluorescent protein” (GFP) is a protein, composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostablility and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm In other embodiments, the virus will carry asensor, for instance a fluorescent activity sensor. A “fluorescent activity sensor” is a fluorescent protein which will alter its fluorescent properties in response to a signal. For instance Ca²⁺ sensors, e.g. yellow cameleon, camgaroo, G-CaMP/Pericam, or TN-L15 will alter their fluorescent properties in the presence of calcium, whereas fluorescent protein voltage sensors, e.g. FlaSh, SPARC, or a VSP, will alter their fluorescent properties in response to changes in the membrane potential. Preferred sensors are VSP1 and/or TN-L15.

Channelrhodopsins are a subfamily of opsin proteins that function as light-gated ion channels. They serve as sensory photoreceptors in unicellular green algae, controlling phototaxis, i.e. movement in response to light. Expressed in cells of other organisms, they enable the use of light to control intracellular acidity, calcium influx, electrical excitability, and other cellular processes. At least three channelrhodopsins are currently known: Channelrhodopsin-1 (ChR1), Channelrhodopsin-2 (ChR2), and Volvox Channelrhodopsin (VChR1). Moreover, some modified/improved versions of these proteins also exist. All known Channelrhodopsins are unspecific cation channels, conducting H+, Na+, K+, and Ca2+ ions. Halorhodopsin is a light-driven ion pump, specific for chloride ions, and found in phylogenetically ancient “bacteria” (archaea), known as halobacteria. It is a seven-transmembrane protein of the retinylidene protein family, homologous to the light-driven proton pump bacteriorhodopsin, and similar in tertiary structure (but not primary sequence structure) to vertebrate rhodopsins, the pigments that sense light in the retina. Halorhodopsin also shares sequence similarity to channelrhodopsin, a light-driven ion channel. Halorhodopsin contains the essential light-isomerizable vitamin A derivative all-trans-retinal. Halorhodopsin is one of the few membrane proteins whose crystal structure is known. Halorhodopsin isoforms can be found in multiple species of halobacteria, including H. salinarum, and N. pharaonis. Much ongoing research is exploring these differences, and using them to parse apart the photocycle and pump properties. After bacteriorhodopsin, halorhodopsin may be the best type I (microbial) opsin studied. Peak absorbance of the halorhodopsin retinal complex is about 570 nm. Recently, halorhodopsin has become a tool in optogenetics. Just as the blue-light activated ion channel channelrhodopsin-2 opens up the ability to activate excitable cells (such as neurons, muscle cells, pancreatic cells, and immune cells) with brief pulses of blue light, halorhodopsin opens up the ability to silence excitable cells with brief pulses of yellow light. Thus halorhodopsin and channelrhodopsin together enable multiple-color optical activation, silencing, and desynchronization of neural activity, creating a powerful cellular and/or neuroengineering toolbox.

In some embodiments using e.g. neurons, a fluorescent activity sensor will be introduced into the cell under the control of a specific promoter and the promoter will be activated by a ligand brought into the cell by another virus, e.g. a transsynaptic virus.

The virus used in the invention may carry a nucleic acid that encodes the desired gene sequence of e.g. a label, a sensor or any gene product the target cell should produce. The virus may comprise elements capable of controlling and/or enhancing expression of the nucleic acid. The virus may be a recombinant virus. The recombinant virus may also include other functional elements. For instance, recombinant viruses can be designed such that the viruses will autonomously replicate in the target cell. In this case, elements that induce nucleic acid replication may be required in a recombinant virus. The recombinant virus may also comprise a promoter or regulator or enhancer to control expression of the nucleic acid as required. Tissue specific promoter/enhancer elements may be used to regulate expression of the nucleic acid in specific cell types. The promoter may be constitutive or inducible.

In some embodiments, the virus will carry genes capable of reprogramming a cell, or influencing its development.

In some embodiments, the method of the invention will be automated and performed by robots.

The cells infected using the present invention may be fixed to a support or in suspension. The cells fixed to a support can be either mono or pluri, layers of cultured cells. The cells can also be embedded in a tissue.

In some embodiments, the attachment of the virus to the support will be reversible. It is however to be noted that any attachment is eventually an equilibrium. Hence, a transfer of the virus to the cell will always eventually happen if one waits long enough. This is especially true in view of the fact that the cells will internalize the viruses and therefore reduce the local concentration of the virus on the surface of the cell. Nevertheless, in order to increase the efficacy of the invention, the strength of the bond linking the virus to the carrier, e.g. the affinity and/or avidity of the molecule binding specifically to a molecule present on the surface of the virus, will be weaker that than the strength of the interaction(s) between the virus and the cell to be infected. The larger this difference will be, the less time will be required to transfer the bound virus from the support onto the cell. In some embodiments, the binding force of the molecule binding specifically to a molecule present on the surface of the virus to one virus will range between 40 pN (picoNewton) and 200 pN. In some embodiments, this force will be less than 50 pN, less than 60 pN, less than 70 pN, less than 80 pN, less than 90 pN, less than 100 pN, less than 110 pN, less than 120 pN, less than 130 pN, less than 140 pN, less than 150 pN, less than 160 pN, less than 170 pN, less than 180 pN, less than 190 pN, or less than 200 pN.

The duration of the contact between the cell and the attached virus will vary, depending on the different strength of interaction. This contact will typically range between less than one second and more than 30 minutes, e.g. 1 second, 5 seconds, 30 seconds, 1 minute, 2 minutes, 5 minutes, 10 minutes, 15 minutes, 20 minutes, 30 minutes, 45 minutes, 1 hour. Although shorter contact times are preferred, some longer time might be required and/or wished, depending on the application.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

To mechanically address viruses to a single cell the inventors chemically functionalized the cantilever of an atomic force microscope (AFM) or a patch pipette made of glass. For functionalization the AFM cantilever or glass pipette were rinsed with Ethanol (EtOH, purity grade) followed by a wash of di-ionized water. After this washing step, the AFM cantilever or glass pipette was dried in a stream of nitrogen. This process was repeated three times. The dried AFM cantilever or glass pipette were then silanized with 5% (3-mercaptopropyl)trimethoxysilane (purum≧97.7%) in toluene. The sulfhydryl-containing silane on the AFM cantilever or glass pipette was reacted with the maleimide group (chemical formula H₂C₂(CO)₂NH) of the cross-linker to form a covalent bond. The N-terminal primary amine of an antibody against the SADΔG-RFP virus (Nature. 2011 Jan. 20; 469(7330):407-10) was then introduced to react with the N-hydroxysuccinimide (NHS, chemical formula C₄H₅NO₃) moiety of the cross-linker (2:1 antibody/cross-linker ratio). Attachment of the viruses to the cantilever or glass pipette was performed by simply dipping said cantilever or glass pipette into a viral solution. The viruses coupled to the AFM cantilever or glass pipette were then mechanically brought into contact with a single cell for a given dwell-time of 120 seconds (AFM cantilever) or 30 minutes (glass pipette). Within this dwell time the virus interacted with the cell surface or after removal of the support, was taken up by the cell and infected it. The inventors tested this “virus-stamping” in cultured baby hamster kidney cells (BHK) and cortical neuron cultures of rat brain. After 2 days for the BHK culture system, respectively 5 days for the cortical neuron culture, the infection by the “stamped” SADΔG-RFP virus infection was monitored using fluorescence microscopy to score RFP expression. After six days each of the six cultures that were stamped by the virus for both BHK and cortical neurons showed one single cell that expressed RFP transferred by the viral gene, thus demonstrating that the cell had funtionnaly taken up the virus and that no diffusion of said virus has taken place during the “stamping” phase.

1. A method of infecting a cell with a virus characterized in that it comprises the step of contacting the cell with a virus attached to a support.

2. The method of claim 1 wherein the virus is attached to the support through a molecule binding specifically to a molecule present on the surface of said virus.

3. The method of claim 2 wherein said molecule binding specifically to a molecule present on the surface of said virus is selected from the group of monoclonal antibody, polyclonal antibody, antibody fragment having a specific binding activity, e.g. F(ab′)2, Fab′, Fab or Fv, chimeric antibody, e.g. humanized antibody, scFv, aptamers and CDRs grafted onto alternative scaffold.

4. The method of claim 2 wherein said molecule binding specifically to a molecule present on the surface of said virus is attached to the support through a linking moiety.

5. The method of claim 4 wherein said linking moiety is selected from the group of polyethyleneglycol (PEG), polypeptide, sugar, nucleic acids, rods and extended fibers, e.g. carbon nanotubes, and combinations thereof.

6. The method of claim 1 wherein said virus is selected from the group of adeno-associated virus, pseudorabies virus, lentivirus, herpes virus and rabies virus.

7. The method of claim 2 wherein said molecule present on the surface of the virus recognized by said specifically-binding molecule is selected from the group of viral coat proteins, or viral lipid molecules and exogenous molecules expressed on the surface of said virus.

8. The method of claim 1 comprising the step of physically touching the cell to be infected with the support to which the virus is attached.

9. The method of claim 8 wherein said support is bound to a micromanipulator.

10. The method of claim 8 wherein said support is the tip of a microelectrode or of a pipette, a bead or microscopic particle, or is a microscopic or nanoscopic device such as a cantilever.

11. The method of claim 1 wherein at least two different viruses are attached to the support contacting the cell.

12. A support to which a molecule binding specifically to a molecule present on the surface of a virus is attached, said molecule binding specifically to a molecule present on the surface of said virus being optionally attached to the support through a linking moiety.

13. The support of claim 11 wherein said molecule binding specifically to a molecule present on the surface of said virus is selected from the group of monoclonal antibody, polyclonal antibody, antibody fragment having a specific binding activity, e.g. F(ab′)2, Fab′, Fab or Fv, chimeric antibody, e.g. humanized antibody, scFv, aptamers and CDRs grafted onto alternative scaffold.

14. The support of claim 11 wherein said viral molecule present on the surface recognized by said specifically-binding molecule is selected from the group of viral coat proteins, or viral lipid molecules and exogenous molecules expressed on the surface of said virus.

15. The support of claim 11, wherein said support is the tip of a microelectrode or of a pipette, a bead or microscopic particle, or is a microscopic or nanoscopic device such as a cantilever.
